(19) United States
(12) Patent Application Publication
(10) Pub. No.: US 2017/0029837 A1
(43) Pub. Date: Feb. 2, 2017

(54) METHOD FOR PRODUCING USEFUL PROTEIN USING PLANT

(71) Applicant: MITSUBISHI CHEMICAL CORPORATION, Chiyoda-ku (JP)

(72) Inventors: Makoto MURASE, Yokohama-shi (JP); Nobuhiro IKEZAWA, Yokohama-shi (JP); Daisuke KITAHARA, Yokohama-shi (JP); Hiroyuki TANAKA, Yokohama-shi (JP)

(73) Assignee: MITSUBISHI CHEMICAL CORPORATION, Chiyoda-ku (JP)

(21) Appl. No.: 15/273,149

(22) Filed: Sep. 22, 2016

Related U.S. Application Data
(63) Continuation of application No. PCT/JP2015/061055, filed on Apr. 9, 2015.

(30) Foreign Application Priority Data
Apr. 10, 2014 (JP) 2014-081110

(51) Int. Cl. C12N 15/82

(52) U.S. Cl. CPC C12N 15/8257; C12N 15/8205

(57) ABSTRACT

Provided is a method for producing a protein with a plant, comprising a step of infecting the plant with Agrobacterium having a polynucleotide encoding a target protein, and a step of expressing the target protein in the plant by further cultivating the plant after infection, wherein production efficiency of the target protein is improved by imparting a damaging stimulus to the roots of the plant.

### TECHNICAL FIELD

The present invention relates to a method for producing a useful protein for use in a medical application and the like efficiently by transient expression using a plant.

### BACKGROUND ART

In recent years, a method for producing a protein using a plant has attracted attention because it enables to express a complex protein, to produce the protein in a large volume and at low cost, and because it facilitates separation and purification while it ensures safety. There have been numerous reports of methods for producing proteins using plants and plant cultivation devices for that purpose, some examples of which are indicated below.

As one example thereof, Patent Document 1 describes a method for producing influenza virus-like particles (VLP) such as those of H1 protein by cultivating Nicotiana benthamiana infected with recombinant Agrobacterium at room temperature. In addition, Patent Document 2 describes a plant obtained by creating a chimeric gene containing a DNA sequence encoding an insecticidal protein under the control of a specific damage-inducible promoter followed by stably incorporating in the genome of a corn plant. This insecticidal protein is locally expressed in damaged tissue directly affected by insect feeding, and imparts resistance to insect feeding to the plant.

On the other hand, Patent Document 3 describes a device for artificially cultivating a plant, and more particularly, describes a root cutting mechanism that cuts the roots of the plant cultivated in a hydroponic cultivation device.

### PRIOR ART DOCUMENTS

### Patent Documents

Patent Document 1: Japanese Unexamined Patent Publication (Translation of PCT Application) No. 2010-533001

Patent Document 2: Japanese Unexamined Patent Publication (Translation of PCT Application) No. 2005-524400

Patent Document 3: Japanese Unexamined Patent Publication No. 2012-50383

### DISCLOSURE OF THE INVENTION

### Problems to be Solved by the Invention

Although technology for producing a protein using a plant is known as described above, the conditions for improving the production efficiency thereof have not been adequately explored.

Namely, although Patent Document 1 describes the detection of influenza virus-like particles (VLP) by transient expression of influenza virus hemagglutinin in a plant induced by Agrobacterium infiltration, the plant is cultivated in solid medium and the processing of roots has not been examined. Consequently, the expression efficiency thereof is presumed to be extremely low.

In Patent Document 2, local high-dose toxin expression is carried out in a plant by environmental stimulation in the form of insect feeding. However, in this method, an insecticidal protein is expressed by incorporating a chimeric gene in the genome of a plant using a specific promoter sequence, and the expression level thereof in undamaged plant tissue is extremely low.

In Patent Document 3, although a method and device are disclosed for severing roots while inhibiting effects on the growth of the plant cultivated with a hydroponic cultivation device, with respect to a plant from which the roots are harvested in the manner of garlic, the object is to repeatedly harvest the root portions of the plant, and not to improve the expression efficiency of a foreign gene introduced into the plant.

An object of the present invention is to improve the production efficiency of a target protein when producing the target protein with a plant by examining processing of the roots of the plant in a step of cultivating the plant and a step of infecting the plant with Agrobacterium.

### Means for Solving the Problems

As a result of conducting extensive studies for solving the aforementioned problems, the inventors of the present invention found a method for significantly improving transient expression efficiency of a protein with a plant using a conventional method by improving cultivation of host plant and processing of the roots of the plant in infection of the plant with Agrobacterium, thereby leading to completion of the present invention.

Namely, the gist of the present invention is as indicated below.

[1] A method for producing a protein with a plant, comprising:

a step of cultivating a plant capable of being infected with Agrobacterium using a hydroponic cultivation method,

a step of infecting the plant grown using a hydroponic cultivation method by contacting with an infecting solution containing Agrobacterium having a polynucleotide encoding a target protein, and

a step of expressing the target protein in the plant by further cultivating the plant after infection; wherein,

a damaging stimulus is imparted to the roots of the plant.

[2] The method for producing a protein described in [1], wherein the damaging stimulus comprises cutting at least a portion of the roots of the plant.

[2-1] The method for producing a protein described in [1] above, wherein the damaging stimulus is at least one damaging stimulus selected from the group consisting of a mechanical stimulus such as damage, a physical stimulus such as a temperature change or pressure change, treatment with a chemical agent and the like, electrical stimulus and radiation stimulus.

[3] The method for producing a protein described in [2], wherein the portion of the roots that is cut is 0.01% by mass to 50% by mass of the total fresh weight of the plant roots.

[4] The method for producing a protein described in [2] or [3], wherein cutting of the roots is carried out prior to the step of infecting.

[4-1] The method for producing a protein described in any of [1] to [3], wherein the damaging stimulus is imparted prior to the step of infecting.

[4-2] The method for producing a protein described in any of [1] to [3], wherein the damaging stimulus is imparted during or after the step of infecting.

[5] The method for producing a protein described in any of [1] to [4], further comprising a step of purifying and/or recovering the target protein from the plant after the step of expressing.

[6] The method for producing a protein described in any of [1] to [5], wherein the target protein is a medicinal protein.

[7] The method for producing a protein described in any of [1] to [6], wherein the plant is Nicotiana benthamiana.

[8] A method for producing a protein with a plant, comprising a step of infecting the plant with Agrobacterium having a polynucleotide encoding a target protein, and a step of expressing the target protein in the plant by further cultivating the plant after infection; wherein,

a damaging stimulus is imparted to the roots of the plant.

[9] The method for producing a protein described in [8], wherein the damaging stimulus is imparted to the roots of the plant prior to infection.

[10] The method for producing a protein described in [8], wherein the damaging stimulus is imparted to the roots of the plant simultaneous to or after infection.

### Effects of the Invention

According to the method for producing a protein with a plant of the present invention, as a result of making contrivances to processing of the roots, expression efficiency of a target protein, or in other words, expression level of the target protein per leaf weight, increases. Consequently, a large amount of the target protein can be efficiently expressed using a comparatively small amount of leaf, thereby contributing to a significant reduction in production costs due to the reduction in the load incurred in a step of purifying and recovering the protein.

### BRIEF DESCRIPTION OF THE DRAWINGS

FIG. 1 is a drawing showing the structure of a plasmid pGFP/MM444.

FIG. 2 is a drawing showing the structure of a plasmid p19/MM444.

FIG. 3A is a schematic representation showing a plant in which a damaging stimulus has not been imparted to the root.

FIG. 3B is a schematic representation showing a plant in which the end of the root has been severed.

FIG. 3C is a schematic representation showing a plant indicating an example of a location where a damaging stimulus other than cutting has been imparted.

### BEST MODE FOR CARRYING OUT THE INVENTION

In a first aspect, the method for producing a target protein with a plant according to the present invention comprises a step of cultivating a plant using a hydroponic cultivation method (cultivation step), followed by a step of infecting the plant with Agrobacterium having a polynucleotide encoding a target protein (infection step), and a step of expressing the aforementioned target protein by cultivating the plant following the infection step (expression step). In addition, in a second aspect, the present invention comprises a step of infecting a plant with Agrobacterium having a polynucleotide encoding a target protein (infection step), and a step of expressing the target protein in the plant (expression step) by further cultivating the plant after the infection step. Here, a damaging stimulus may be imparted to the roots of the plant prior to the infection or simultaneous to or after the infection.

A plant usable in the present invention are a plant capable of being infected with Agrobacterium, and there are no particular limitations thereon provided it is a plant that express a target protein. Examples thereof include dicotyledonous plants and monocotyledonous plants. More specifically, examples of dicotyledonous plants include those of the family Solanaceae such as tobacco, potato and tomato plants, those of the family Brassicaceae such as arugula, turnip greens, potherb mustard, mustard greens or thale cress plants, those of the family Asteraceae such as chicory, endive or artichoke plants, those of the family Fabaceae such as alfalfa, mung bean or soybean plants, those of the family Amblycipitidae such as spinach or sugar beet plants, those of the family Lamiaceae such as perilla or basil plants, and those of the family Apiaceae such as honewort plants. Examples of monocotyledonous plants include those of the family Gramineae such as rice, wheat, barley and corn plants, and those of the family Malvaceae such as cotton plants. Among these, plants belonging to the family Solanaceae are preferable, and tobacco plants are particularly preferable.

Examples of tobacco plants include Nicotiana tabacum, N. benthamiana, N. alata, N. glauca, N. longiflora, N. persica, N. rustica and N. sylvestris. Nicotiana benthamiana is preferable.

In the present invention, there are no particular limitations on the target gene provided it is a gene used for medical or industrial applications. It is preferably a gene used for medical applications.

A medicinal gene is classified into a therapeutic protein and a diagnostic protein, examples of the therapeutic protein include a peptide, vaccine, antibody, enzyme and hormone (and preferably a peptide hormone), and more specifically, examples of a therapeutic protein include a viral protein used as a vaccine, granulocyte colony-stimulating factor (G-CSF), granulocyte macrograph colony-stimulating factor (GM-CSF), a hematopoietic factor such as erythropoietin (EPO) or thrombopoietin, a cytokine such as interferon, interleukin 1 (IL-1) or IL-6, a monoclonal antibody and fragments thereof, tissue plasminogen activator (TPA), urokinase, serum albumin, blood coagulation factor VIII, leptin, insulin and stem cell factor (SCF). In addition, examples of a diagnostic protein include antibody, enzyme and hormone.

Preferable examples of a viral protein used as a vaccine include a component protein of viral-like particles (VLP). a VLP component protein may be a single protein or contain one or more proteins. Examples of a virus include influenza virus, norovirus, human immunodeficiency virus (HIV), human hepatitis C virus (HCV) and human hepatitis B virus (HBV), an example of a VLP component virus of influenza virus is influenza hemagglutinin (HA) protein, and an example of a VLP component virus of norovirus is Norwalk virus capsid protein (NVCP).

An industrial protein refers to a protein used in food, animal feed, cosmetics, fibers, cleaners or chemicals, and examples thereof include a peptide, enzyme and functional protein. More specifically, examples include proteinase, lipase, cellulase, amylase, peptidase, luciferase, lactamase, collagen, gelatin, lactoferrin and jellyfish green fluorescent protein (GFP).

The following provides an explanation of each step of the production method of the present invention.

<Cultivation Step>

In one embodiment thereof, the present invention is characterized by having a step of cultivating the aforementioned plant using a hydroponic cultivation method.

Hydroponic cultivation refers to nutrient solution cultivation, and consists of dynamic cultivation, in which the nutrient solution (liquid fertilizer) flows around roots of a plant, and static cultivation, in which the flow of nutrient solution is dependent on capillary action. Dynamic cultivation consists of a nutrient film technique (NFT), in which nutrient solution flows over a gently inclined flat surface in the form of a thin film, and a deep flow technique (DFT), in which a plant is cultivated in a pooled nutrient solution. The deep flow technique (DFT) promotes nutrient absorption by providing an adequate supply of oxygen to the roots due to the flow of liquid fertilizer, and further stabilizes the rhizosphere environment, including temperature and concentration of the liquid fertilizer, thereby offering the advantage of providing a constant cultivation environment.

Hydroponic cultivation is preferable since it facilitates the use of multiple cultivation shelves, recycling of nutrient liquid, and management of fertilizer components and pH, and among these, the bare-root technique, in which the roots are exposed in the nutrient liquid, is preferable. The bare-root technique refers to a technique for cultivating all or a portion of the roots while directly immersed in the nutrient solution. Furthermore, the bases of the roots may be supported with a support such as urethane. Since the roots are able to freely extend in the water resulting in increased contact surface area with the nutrient solution, they are able to absorb adequate amounts of water and nutrients, thereby resulting in more vigorous growth than ordinary soil cultivation.

In addition, the number of days of cultivation in the cultivation step is normally 5 days or more, preferably 7 days or more and more preferably 10 days or more, and normally 35 days or less, preferably 28 days or less and more preferably 21 days or less.

In the cultivation step, replanting may be carried out as necessary. The time of replanting is preferably 6 days to 15 days after the start of cultivation.

Furthermore, in the production method of the present embodiment, a seedling growth step is preferably carried out prior to the cultivation step. The seedling growth step refers to a step of allowing plant seedlings to germinate and grow in an artificial environment for a fixed period of time, after which the seedlings are replanted in the cultivation step. Temperature, humidity and other conditions in the seedling growth step can be the same as conditions in the aforementioned cultivation step. In addition, although ordinary conditions such as the use of sunlight, fluorescent light, LED, cold cathode fluorescent lamp (CCFL, HEFL) or inorganic/organic EL can be employed as conditions for illumination in the seedling growth step, the seedlings are preferably grown using a light/dark cycle in which the duration of illumination is from 12 to 24 hours per day. Furthermore, the duration of illumination being 12 to 24 hours per day means that illumination is not necessarily required to be provided continuously, and in the case of a duration of illumination of 20 hours per day, for example, 10 or more hours of continuous illumination may be carried out twice per day.

There are no particular limitations on the plant production system used in the cultivation step, and any system may be used provided it allows cultivation to be carried out under the aforementioned conditions. A semi-closed or closed plant factory is preferable in consideration of the ease of adjusting the light wavelength and intensity during cultivation, and a closed plant factory is more preferable. Examples of semi-closed types include horticultural facilities and sunlight-type plant factories.

Here, a closed plant factory refers to a plant factory that is not exposed to sunlight, and is a system used to cultivate a plant in a space for which temperature, humidity, carbon dioxide concentration, artificial light wavelength and illumination time are controlled. Since the use of a closed plant factory enables environmental control, it has the effect of stabilizing the quality of the plant and substances produced thereby, as well as the effect of being able to prevent infection by pathogenic bacteria contained in outside air.

An example of a closed plant factory is a system that contains an environmentally-controlled room, a plant cultivation vessel shelf installed in the environmentally-controlled room on which are placed plant cultivation vessels, and lighting arranged near the plant cultivation vessel shelf that radiates light onto the plant in close proximity thereto. A plurality of plant cultivation vessel shelves can be arranged.

A plant cultivated in the cultivation step preferably has a plant height (cm) of 2 cm or more, more preferably 3 cm or more, preferably 25 cm or less and more preferably 15 cm or less. Plant height within the aforementioned ranges is advantageous for improving space time yield (STY) of the closed plant factory since it allows the use of multiple cultivation shelves. Moreover, this also facilitates precise control of cultivation environmental conditions such as temperature, humidity and air flow, and allows to obtain the effect of improving plant growth rate in the cultivation step and expression of the target protein in the expression step, thereby making this preferable. Furthermore, “plant height” as referred to here refers to the length from the lower end of the above ground portion to the growing point of the plant, and can be determined by measuring the length of plant height after having removed the below ground portion of the plant immediately after harvesting.

The fresh weight (g) of the above ground portion of a plant cultivated in the cultivation step is preferably 3 g or more, more preferably 10 g or more, preferably 100 g or less and more preferably 70 g or less. A plant in which the fresh weight of the above ground portion is within the aforementioned ranges is demonstrated to have a rapid growth rate, and if the plant is used at the time of this rapid growth rate, the production efficiency of the target protein is improved, thereby making this preferable.

The leaf weight of a plant cultivated in the cultivation step is preferably 2.5 g or more, more preferably 7.5 g or more, preferably 80 g or less and more preferably 60 g or less. A plant in which the leaf weight thereof is within the aforementioned ranges is demonstrated to have a rapid growth rate, and if the plant is used at the time of this rapid growth rate, the production efficiency of the target protein is improved, thereby making this preferable.

Although there is no particular limitation on cultivation conditions in the cultivation step provided they are suitable for plant growth and production of a target protein, a plant can be cultivated, for example, under the conditions indicated below.

The temperature in the plant factory is normally 10° C. or higher, preferably 15° C. or higher, normally 40° C. or lower and preferably 37° C. or lower.

The humidity in the plant factory is normally 40% or higher, preferably 50% or higher, normally 100% or lower and preferably 95% or lower.

The carbon dioxide concentration in the plant factory is normally 300 ppm or more, preferably 500 ppm or more, normally 5000 ppm or less and preferably 3000 ppm or less.

Although there are no particular limitations thereon, examples of the light source used in the cultivation step include sunlight, fluorescent lamp, LED, cold cathode fluorescent lamp (CCFL, HEFL) and inorganic/organic EL. Preferable examples include a fluorescent lamp, LED and cold cathode fluorescent lamp, and an LED is particularly preferable. LEDs are preferable since they demonstrate high light conversion efficiency while saving on energy in comparison with an incandescent light bulb or HID lamp. In addition, they are also preferable from the viewpoint of releasing only a small amount of heat rays that cause leaf scorching in the plant.

Light intensity in the cultivation step can be evaluated by measuring photosynthetic photon flux density (PPFD). PPFD represents the number of photons per unit time and unit area of light in the visible range of 400 nm to 700 nm that is effective for photosynthesis, and is in units of μmol·m⁻²·s⁻¹. A plant are cultivated at a PPFD of normally 30 μmol·m⁻²·s⁻¹ to 600 μmol·m⁻²·s⁻¹, preferably 50 μmol·m⁻²·s⁻¹ to 500 μmol·m⁻²·s⁻¹, and more preferably 70 μmol·m⁻²·s⁻¹ to 400 mol·m⁻²·s⁻¹. Here, PPFD can be measured using a photon meter and the like.

Furthermore, it is not necessary to use the aforementioned conditions for the light intensity conditions through the entire cultivation step, but rather a plant may be cultivated while using the aforementioned conditions only during a fixed period of the cultivation step, such as by dividing the cultivation step into a first half and second half and using the aforementioned intensity conditions for only the second half of the cultivation step. In this case, the period during which the aforementioned conditions are used preferably constitutes 1% or more, and more preferably constitutes 20% or more, of the entire cultivation period.

There are no particular limitations on light intensity conditions during the period among the entire duration of the cultivation step in which the aforementioned conditions are not used for the light intensity conditions, and a plant may be cultivated under sunlight in an open plant factory during this period.

In addition, a plant is preferably cultivated in the cultivation step using a light-dark cycle in which the duration of illumination is 10 hours to less than 24 hours per day, or cultivated under continuous illumination. Among these, cultivation under continuous illumination is preferable. If illumination is within the aforementioned range, plant growth rate is accelerated and the duration of cultivation until harvest is shortened, thereby making this preferable. Furthermore, the duration of illumination being 10 hours to less than 24 hours per day means that continuous illumination is not necessarily required, and in the case of a duration of illumination of 20 hours per day, for example, 10 or more hours of continuous illumination may be carried out twice per day.

Furthermore, the light used for illumination here may be pulsed light. Pulsed light is obtained by flashing an LED and the like at a short interval of 1 microsecond to 1 second, and as a result of using such pulsed light, since light can be prevented from shining on the plant during the time light is not required physiologically, and can be shined on the plant only during the time light is required, the rate of photosynthesis can be increased and electrical power costs can be reduced. Illumination time in this case is the total length of time the pulsed light is illuminated per day based on the premise of including the length of time the LED is not on in the illumination time.

<Infection Step>

In the infection step, a plant obtained in the aforementioned cultivation step is infected with Agrobacterium having a polynucleotide that encodes a target protein. Agrobacterium infection is a superior method for introducing recombinant DNA into plant cells, and is preferably used in the present invention.

The polynucleotide that encodes the target protein refers to the polynucleotide that encodes the target medicinal or industrial protein as described above. A polynucleotide to which mutations or alterations have been suitably added to a naturally-occurring sequence within a range that can obtain a desired target protein may also be used as polynucleotide.

In order to overly express a polynucleotide encoding a target protein in a plant, the polynucleotide is functionally linked downstream from a suitable promoter and the resulting polynucleotide construct is introduced into a plant cell using the Agrobacterium method. Examples of the aforementioned promoter include, but are not limited to, the 35S promoter of cauliflower mosaic virus (CaMV) and maize ubiquitin promoter. The aforementioned promoter is referred to as a constitutive promoter or constitutive regulatory promoter, and continuously expresses target protein throughout the entire life of the plant in various parts throughout the plant. The term “constitutive” indicates that, although a gene under the control thereof is not necessarily required to be expressed at the same level in all tissues and cells, it is expressed over a wide range of plant tissues and cells, and is preferably used in the method of the present invention.

In one embodiment of the present invention, a tissue-specific promoter or inducible promoter may be used, and these include mesophyll-specific promoters and inducible promoters induced by light, heat, shock, low temperatures or water stress and the like.

When carrying out the Agrobacterium method, a binary vector or intermediate vector can be used that contains transfer DNA (T-DNA) derived from a Ti plasmid or Ri plasmid of Agrobacterium (Nucl. Acids Res., 12(22):8711-8721 (1984); Plasmid, 7, 15-29 (1982)). Specific examples of binary vectors include, but are not limited to, pBI vectors (such as pRiceFOX), pCAMBIA vectors (vector skeleton: pPZP vector), and pSMA vectors (Plant Cell Reports, 19:448-453 (2000)).

As for the particulars of the infection step, infection is carried out by immersing at least a portion of the aforementioned plant in an infecting solution containing the aforementioned Agrobacterium and adjusting pressure while in that state. The infecting solution containing Agrobacterium used is obtained by suspending microbial cells, obtained by culturing Agrobacterium that has been transformed by the aforementioned vector, in a buffer solution suitable for infiltration into plant tissue, and the turbidity of the infecting solution at an OD value of 600 is preferably about 0.05 to 5, more preferably about 0.1 to 2 and even more preferably about 0.2 to 1.

The immersion of the plant does not require that the entire plant be immersed in the infecting solution, but rather a portion of the plant, such as the stem or roots, may be sticking out of the infecting solution.

Adjusting the pressure while the plant is immersed in the infecting solution refers to infecting plant cells with Agrobacterium by using at least one type of pressure cycling treatment selected from pressurization treatment and depressurization treatment with a portion of the plant immersed in the infecting solution containing Agrobacterium. In the case of carrying out pressurization treatment in which treatment is carried out at a pressure higher than atmospheric pressure (normally, 1 atmosphere=101.325 kPa=approx. 0.1 MPa), a plant is treated at a pressure of at least 1.1 atm (112 kPa), 1.5 atm (152 kPa), 2 atm (203 kPa), 2.5 atm (253 kPa), 3 atm (304 kPa), 4 atm (405 kPa), 5 atm (507 kPa) or any pressure between them or any pressure higher than them. Pressure is preferably within the range of 1.7 atm (172 kPa) to 10 atm (1013 kPa) and more preferably within the range of 4 atm (405 kPa) to 8 atm. In the case of carrying out depressurization treatment in which treatment is carried out at a pressure lower than atmospheric pressure, pressure is preferably within the range of 0.005 atm (0.5 kPa) to 0.3 atm (30 kPa), more preferably within the range of 0.01 atm (1.0 kPa) to 0.1 atm (10.1 kPa), and even more preferably within the range of 0.02 atm (2.0 kPa) to 0.06 atm (6.1 kPa). In the case depressurization pressure is excessively high, infiltration of infecting solution becomes inadequate, thereby making this undesirable. Conversely, in the case the pressure is excessively low, the solution may reach its boiling point and evaporate rapidly resulting in loss of liquid and inadequate infiltration and the scale of the production process and equipment may become excessively large, thereby making this also undesirable. Thus, although the duration in which this pressurization treatment and depressurization treatment are carried out can be suitably set corresponding to the type of plant and treated tissue, they are about 10 seconds to 10 minutes, preferably 20 seconds to 5 minutes and even more preferably 30 seconds to 3 minutes.

When adjusting pressure with a plant immersed in the infecting solution, depressurization treatment is preferable to pressurization treatment since it is superior in terms of the device being comparatively simple and greater convenience. Vacuum infiltration is one example of a form of depressurization treatment, the plant is preferably infected with Agrobacterium using this vacuum infiltration method, and a transient expression method based on vacuum described in Plant Science, 122, 1:101-108 (1997) is used more preferably.

Vacuum infiltration refers to a method for enabling infiltration of Agrobacterium between plant cells or into the interstitial space thereof wherein the vacuum physically generates negative atmospheric pressure that causes a reduction in the space between cells in plant tissue. The air space in plant tissue becomes less, the longer the continuation period or the lower the pressure of the vacuum. The infecting solution (containing Agrobacterium having a transformation vector) is able to move into plant tissue by increasing the pressure. A vacuum can be applied for a fixed period of time to a portion of the plant in the presence of Agrobacterium in order to infect the plant. After having reduced pressure to achieve a vacuum state, the plant can be infected by returning to normal pressure (pressure recovery). As a result of infection, Agrobacterium containing a polynucleotide construct enters plant tissue such as a leaf or other aboveground portion of the plant (including the stem, leaf or flower), another portion of the plant (such as the stem, root or flower) or any intercellular space throughout the plant. Agrobacterium infects the plant after passing through the epidermis and then transfers the polynucleotide to plant cells. The polynucleotide is transcribed as an episome and then translated to mRNA, and although this brings about production of a target protein in the infected cells, polynucleotide multiplies in the nucleus transiently.

<Expression Step>

In the expression step, a plant that has completed the infection step is cultivated to express the target protein. Although there are no particular limitations on the cultivation conditions in the expression step provided they are conditions that enable the target protein to be efficiently expressed, conditions such as temperature and humidity in the expression step can be the same conditions as the conditions in the aforementioned cultivation step.

In addition, ordinary conditions such as the use of sunlight, fluorescent light, LED, cold cathode fluorescent lamp (CCFL, HEFL) or inorganic/organic EL can be employed as conditions for illumination. The number of days of cultivation in the expression step is preferably 3 days or more, more preferably 4 days or more, preferably 14 days or less and more preferably 10 days or less.

<Processing of Roots for Enhancing Protein Expression>

The method of the present invention is characterized by imparting a damaging stimulus to the roots of a plant at any stage in the aforementioned cultivation step, infection step and expression step. Although there are no particular limitations on the timing when the damaging stimulus is imparted, the damaging stimulus is preferably imparted so that an adequate length of time is allocated for the polynucleotide encoding the target protein to be transcribed and translated and subsequently expressed in the plant cells after having undergone treatment for imparting this stimulus. Consequently, it is preferable to impart the damaging stimulus at the stage in which the plant is suitably grown in the cultivation step, and particularly at the stage prior to contacting the plant with infecting solution containing Agrobacterium or during the time immediately thereafter. More preferably, the stimulus can be imparted to the roots concurrently to carrying out the procedure for immersing the plant in the infecting solution containing Agrobacterium. Alternatively, such treatment may be carried out during the initial stage of the aforementioned infection step or expression step. More particularly, treatment may be carried out, for example, during the time from 24 hours prior to carrying out infection until immediately prior to infection or treatment may be carried out during the time up to within 72 hours after infection, and treatment is preferably carried out up to 12 hours prior to carrying out infection, and is more preferably carried out by 1 hour before and preferably 30 minutes before infection.

In addition, the aforementioned cultivation step, infection step and expression step may each be carried out in separate plant farms and the like. Moreover, a commercially available cultivated plant may be purchased and then used to carry out the infection step and expression step. At that time, the purchased plant may immediately be subjected to damaging stimulus and infection with Agrobacterium, or these treatments may be sequentially carried out after a prescribed length of time.

As was described above, a plant cultivated using the bare-root technique can be preferably used in the method of the present invention. However, since a plant cultivated using the bare-root technique has all or a portion of its roots in an exposed state, it becomes easy to suitably impart a damaging stimulus to the roots in each step, thereby making this preferable. For example, a damaging stimulus can be imparted at an intermediate point in the cultivation step or expression step without having to carry out a procedure for removing the roots from soil or a solid medium, thereby enabling cultivation to be further continued. In addition, since various procedures are normally carried out in the infection step on the leaf portions of a plant with the roots in an exposed state, a procedure for imparting a damaging stimulus to the roots of the plant can be carried out simultaneous to these procedures, thereby making it possible to shorten the length of time required for the steps of entire production method.

On the other hand, in the case of using a plant cultivated by hydroponic cultivation and particularly the bare-root technique, since cultivation is not likely to be accompanied by contaminants or contaminating bacteria derived from soil surrounding the roots, there is the advantage of making it easier to process the roots when carrying out a procedure for imparting a damaging stimulus. In the case of immersing in an infecting solution and adjusting the pressure in particular, it is less likely to have any problem such as contamination, even if the entire plant, including the roots, is arranged within the pressure device, thereby making this preferable.

In the present invention, a “damaging stimulus” includes at least one type of environmental stimulus selected from the group consisting of a mechanical stimulus such as damage, a physical stimulus such as a temperature change or pressure change, treatment with a chemical agent and the like, electrical stimulus and radiation stimulus, and although this typically includes cutting at least a portion of the roots, this damaging stimulus is not limited thereto. For example, this includes imparting damage to a portion of plant tissue in a form such that the function of plant roots is partially inhibited, imparting an open wound using a sharp protruding object, or imparting a closed wound not having an opening in the manner of a bruise using a blunt object, and the roots are not necessarily required to be completely severed from the plant.

The following provides an example of a method for imparting a damaging stimulus using the drawings. FIG. 3A is a schematic representation showing a plant prior to imparting a damaging stimulus. In FIG. 3A, the entire root is in an exposed state. In FIG. 3B, a portion of the end of the root is severed and the severed root is separated from the plant. In FIG. 3C, the location where an open or closed wound is imparted in the vicinity of the base of a plant root is surrounded by a broken line, while the location of the resulting linear wound is indicated with a dotted line.

There are no particular limitations on the methods used to sever the roots or impart a stimulus, and the roots may be severed or damaged with a sharp tool such as scissors or knife, or may be severed or damaged by physical stress such as bending fracture or shear fracture.

Although there are also no particular limitations on the extent of the range over which the roots are severed or damaged, the roots can be severed or damaged within a range that does not cause a decrease in expression efficiency of the target protein as a result of excessive impairment of absorption of water and nutrients by the roots. With respect to the location where the roots are severed or damaged, although the roots may be severed or damaged at the end of the roots or at the portion where the roots join the stem, at least about 0.01% by mass of the roots are preferably severed or damaged from the apices of the roots based on the total fresh weight of the roots. An upper limit of the severed amount or damaged amount of about 50% by mass of the total fresh weight of the roots is considered not to present a problem for the reasons described above. Here, the selection of those roots which are severed or damaged may be such that all of the roots are severed or damaged by roughly the same amount, an arbitrary number of roots among a plurality of roots may be subjected to a prescribed amount of severing or damage, or severing or damage may be adjusted so as to be within the aforementioned range based on the total fresh weight of the roots.

In a preferred embodiment of the present invention, the portion of the roots that is severed or damaged is 0.1% by mass to 40% by mass, preferably 1% by mass to 30% by mass, and more preferably 2% by mass to 20% by mass, based on the total fresh weight of the roots of the aforementioned plant.

Although the reason why the expression efficiency of a target protein improves as a result of severing or damaging the roots of a plant in this manner is not necessarily clear, it is presumed that as a result of the roots being damaged, absorption of nutrients decreases, physiological activity of the plant is prioritized to repairing the damage while the supply of nutrients to leaves is reduced, a mechanism for generating resistance to over expression of a foreign gene is weakened due to activation or deactivation of a specific control mechanism due to transmission of some form of signal, or the plant becomes easily infected by Agrobacterium. In the method of the present invention, the expression efficiency of a target protein is thought to improve regardless of the type of target protein.

<Target Protein Recovery Step>

In the expression step, a target protein that has accumulated in a plant is preferably recovered from the plant. A fraction containing the target protein is preferably acquired from the plant followed by purifying the target protein using a suitable method. Furthermore, the polynucleotide encoding the target protein may contain a tag sequence for the purpose of purification.

In the method of the present invention, only the expressed amount of target protein increases without any increase in the overall weight of the plant. The target protein can be recovered and purified using a starting material having a high content, which is thought to make it possible to reduce the load on the purification step that accounts for a large proportion of production costs.

### EXAMPLES

Although the following provides a more detailed explanation of the present invention by indicating examples thereof, the present invention is not limited in any way by these examples.

### 1. Preparation of Plant Biomass

(1) Seeding

0.78 g/L of seeding liquid fertilizer (Otsuka House No. 1 (Otsuka Agritechno Co., Ltd.) and 0.25 g/L of Otsuka House No. 2 (Otsuka Agritechno Co., Ltd.) were soaked into a urethane mat for hydroponic cultivation (Ematsu Chemical Co., Ltd., 587.5 mm (W)×282 mm (D)×28 mm (H), 12×2 receptacles, diameter: 9 mm) followed by housing in a seedling tray (600 mm (W)×300 mm (D)×300 mm (H)) and seeding with seeds of Nicotiana benthamiana.

(2) Seedling Growth

Following seeding, the plants were grown for 12 days in an artificial plant growth chamber (NC-410HC, Nippon Medical & Chemical Instruments Co., Ltd.) at a temperature of 28° C. and light/dark cycle of 16 hours of light and 8 hours of darkness.

(3) Cultivation (First Stage)

The urethane mat used for growing the seedlings was separated into individual receptacles and replanted to a cultivation (first stage) panel (600 mm (W)×300 mm (D), 30 holes). Following replanting, the cultivation (first stage) panel was placed in a cultivation device, and the plants were cultivated for 9 days using the deep flow technique. Environmental conditions and liquid fertilizer conditions were controlled as indicated below.

<Environmental Conditions>

Temperature: 28° C.

Relative humidity: 60% to 80%

CO₂ concentration: 400 ppm

Lighting: Average photosynthetic photon flux density (PPFD): 140 μmol/m²·sec, 24 hour continuous illumination, three-wavelength fluorescent lamp (Lupica Line, Mitsubishi Electric Corp.)

<Liquid Fertilizer Conditions>

Liquid fertilizer consisting of Liquid Fertilizer Solution A (Otsuka House No. S1, 150 g/L, Otsuka House No. 5 (Otsuka Agritechno Co., Ltd., 2.5 g/L) and Liquid Fertilizer Solution B (Otsuka House No. 2, 100 g/L) were respectively dissolved in dechlorinated water and used by mixing equal volumes of each solution. pH Adjuster Down (Otsuka Agritechno Co., Ltd.) and 4% aqueous KOH solution were used to adjust pH. The electrical conductivity (EC) and pH of the liquid fertilizer were adjusted to an EC of 2.3 mS/cm and pH 6.0 using “Easy Treatment Fertilizer Controller 3” (CEM Corp.).

(4) Cultivation (Second Stage)

The plant bodies were removed from the cultivation (first stage) panel and planted in a cultivation (second stage) panel (600 mm (W)×300 mm (D), 6 holes). After replanting, the cultivation (second stage) panel was placed in a cultivation device, and the plants were cultivated for 7 days (28 days after seeding) using the deep flow technique. The liquid fertilizer conditions were controlled to the same liquid fertilizer conditions as first stage cultivation with the exception of changing the electrical conductivity (EC) to 4.0 mS/cm.

<Environmental Conditions>

Temperature: 28° C.

Relative humidity: 60% to 80%

CO₂ concentration: 400 ppm

Lighting: Average photosynthetic photon flux density (PPFD): 140 μmol/m²·sec, 24 hour continuous illumination, three-wavelength fluorescent lamp (Lupica Line, Mitsubishi Electric Corp.)

### 2. Preparation of Transformed Agrobacterium

(1) Expression Plasmids

The following two types of expression plasmids were used to examine jellyfish green fluorescent protein (GFP) expression.

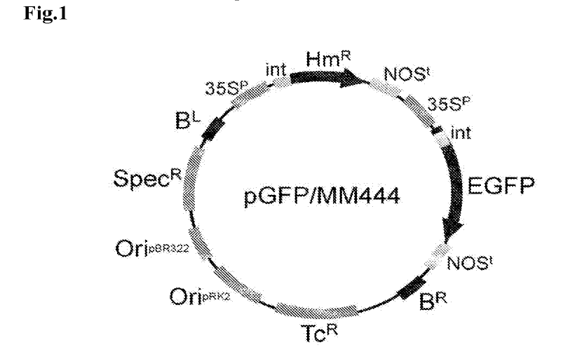

A kanamycin resistance expression cassette (consisting of nopaline synthase gene promoter, kanamycin resistance gene and nopaline synthase gene terminator) of plant binary vector pMM444 (Japanese Unexamined Patent Publication No. H9-313059) was replaced with a hygromycin resistance expression cassette consisting of the 35S promoter of cauliflower mosaic virus, the first intron of castor oil plant catalase gene, hygromycin resistance gene and nopaline synthase gene terminator) derived from pIZI (Japanese Unexamined Patent Publication No. H7-274752). Into the plasmid obtained in this manner, an EGFP expression cassette was further introduced, in which the β-glucuronidase gene of a GUS expression cassette (consisting of 35S promoter of cauliflower mosaic virus, the first intron of castor oil plant catalase gene, β-glucuronidase gene, and nopaline synthase gene terminator) derived from pIG221 (Plant Cell Physiol., 31, 805 (1990)) was replaced with EGFP gene (pEGFP-N3, Clontech Laboratories, Inc.), to create an EGFP gene expression plasmid (to be referred to as “pGFP/MM444” and its structure is shown in FIG. 1).

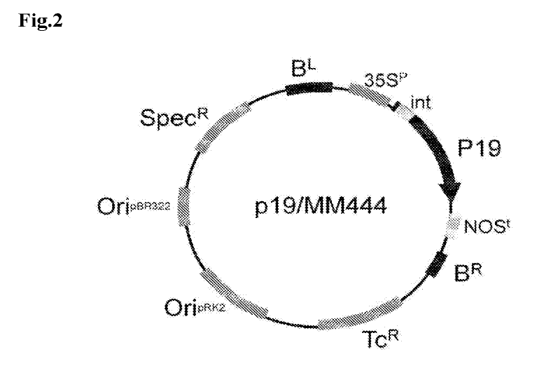

In addition, the hygromycin resistance expression cassette in pGFP/MM44 was deleted and the EGFP gene of the EGFP expression cassette was replaced with P19 gene to create a P19 gene expression plasmid (to be referred to as “p19/MM444” and its structure is shown in FIG. 2). The P19 gene functions to enhance expression of EGFP gene, and this p19/MM444 was used for co-expression with pGFP/MM444.

The abbreviations for the genes and their control regions in FIGS. 1 and 2 stand for:

35S^{P}: Cauliflower mosaic virus 35S promoter

int: Castor oil plant catalase gene, first intron

Nos^{t}: Nopaline synthase gene terminator

Spec^{R}: Spectinomycin resistance gene

Tc^{R}: Tetracycline resistance gene

Hm^{R}: Hygromycin resistance gene

ori^{pBR322}: pBR322 ori

Ori^{pRK2}: pRK2 ori

B^{L}: T-DNA left border

B^{R}: T-DNA right border

(2) Agrobacterium Transformation and Preparation of Transformed Agrobacterium Glycerol Stock

The aforementioned plasmids (pGFP/MM444 and p19/MM444) were respectively introduced into Agrobacterium strains (Agrobacterium tumefaciens AGL1: Rhizobium radiobacter ATCC BAA-10, American Type Culture Collection (ATCC), Manassas, Va. 20108, USA) by electroporation (Mattanovich et al., 1989) (the resulting transformed Agrobacterium are referred to as GFP-Agrobacterium and P19-Agrobacterium, respectively).

The transformed Agrobacterium (GFP-Agrobacterium and P19-Agrobacterium) were cultured in LB medium containing 25 μg/ml of carbenicillin and 50 μg/ml of spectinomycin (Sigma-Aldrich Corp.), followed by the addition of glycerol to prepare to a final glycerol concentration of 30% by mass and then storing at −80° C. to prepare glycerol stocks of each transformed Agrobacterium.

(3) Preparation of Transformed Agrobacterium for Use in the Infection Step

Glycerol stocks of the transformed Agrobacterium prepared in Section (2) above (GFP-Agrobacterium and P19-Agrobacterium) were inoculated into the LB medium and cultured.

After culture, the bacterial cells were collected by centrifugation and the resulting cells were suspended in infiltration buffer (5 mM MES, 10 mM MgCl₂, pH 5.6) to obtain a solution with concentrated bacteria. The resulting concentrated bacteria were added to 4 L of infiltration buffer so that the OD 600 value of a 1:1 bacterial liquid mixture of GFP-Agrobacterium and P19-Agrobacterium was 0.8 the pH was adjusted to 5.6 to obtain an Agrobacterium bacterial solution for use in the infection step.

### Examples 1 and 2 and Comparative Example 1

### 3. Infection by Vacuum Infiltration

### Infection Step

The roots of Nicotiana benthamiana obtained in Section 1 (4) above were respectively not cut (0% by mass, Comparative Example 1, see FIG. 3A), were cut at a ratio based on total root weight of 2% by mass (Example 1, see FIG. 3B) or were cut at a ratio of 18% by mass (Example 2, see FIG. 3B) from the root apices thereof on day 28 after seeding. The plants were each immediately (within 30 minutes after cutting) inverted and submerged in Agrobacterium bacterial solution (prepared in Section 2 (3) above) contained in a beaker so that the above-ground portion was immersed in the solution. Subsequently, the beakers were placed in a vacuum desiccator (FV-3P, Tokyo Glass Kikai Co., Ltd.) and depressurized by allowing to stand still for 1 minute at 19 Torr to 40 Torr. Subsequently, the valves were opened all at once to return to the normal atmospheric pressure.

### 4. Cultivation of Infected Leaves

### Expression Step

After infection, cultivation was carried out using an artificial plant growth chamber (Nippon Medical & Chemical Instruments Co., Ltd.). The plants were cultivated using a three-wavelength fluorescent lamp (Lupica Line®, Mitsubishi Electric Corp.) at a light/dark cycle consisting of 16 hours of light and 8 hours of darkness and average PPFD of 150 μmol/m²·sec. The liquid fertilizer had electrical conductivity (EC) of 2.1 mS/cm to 2.2 mS/cm and the pH was 5.5. The temperature was cycled at 25° C. when light and 20° C. when dark. In addition, relative humidity was controlled to 60% to 85%. The Agrobacterium-infected Nicotiana benthamiana, on which the aforementioned expression step was carried out over the course of 6 days, was harvested of all leaves excluding the leaf stalks. The leaves were subsequently stored at −80° C.

### 5. Measurement of GFP Expression Levels

(1) Preparation of Crude Extracts

The Agrobacterium-infected leaves for expression of GFP and p19 placed in frozen storage in Section 4 above were transferred to a mortar and ground up in liquid nitrogen. Subsequently, GFP assay extraction buffer (50 mM Tris-HCl, 150 mM NaCl, 2 mM EDTA, 0.1% Triton-X100 (pH 7.25)) equal to 6 times the fresh weight of the sample was added and vigorously shaken to carry out protein crude extraction. 1 ml of the crude extract was transferred to a 1.5 ml Eppendorf tube and centrifuged for 10 minutes at 400×g. The supernatant was recollected and subjected to the GFP quantitation described below.

(2) GFP Quantitation

Detection of GFP fluorescence was carried out by using the Wallac ARVO SX 1420 Multilabel Counter (Perkin-Elmer Life Sciences Inc.), and fluorescence at 507 nm generated by excitation light at 485 nm was detected. Serially diluted GFP standards (rAcGFP1 Protein, Takara Bio Inc.) were used for the quantitation. The measurement samples were diluted 5-fold with GFP extraction buffer, 100 μL aliquots were dispensed into a 96-well microtiter plate (Nunc FluoroNunc Plate, Thermo Fisher Scientific K.K.), measurement results for three strains were averaged, and GFP expression levels per leaf fresh weight (mg/kg-FW), that is, expression efficiency, are shown in Table 1 based on a value of 100% for the case of plants for which the roots were not cut.

According to Table 1, GFP expression levels per leaf fresh weight with the plants whose roots were cut increased in comparison with plants whose roots were not cut. As a result, expression efficiency was determined to improve by cutting the roots.

TABLE 1

| | | GFP Expression Level |
|---|---|---|
| | Root Processing | per Leaf Fresh Weight |
| Comparative | Roots were not cut | 100.0% |
| Example 1 | | |
| Example 1 | Portion of roots cut from the apices | 119.1% |
| | was 2% of total root weight | |
| Example 2 | Portion of roots cut from the apices | 119.4% |
| | was 18% of total root weight | |

### Comparative Example 2

Nicotiana benthamiana obtained in Section 1 (4) above was inverted on day 28 after seeding and submerged in Agrobacterium bacterial solution (prepared in Section 2 (3) above) contained in a beaker so that the above ground portion was immersed in the solution. Subsequently, the beaker was placed in a vacuum desiccator (FV-3P, Tokyo Glass Kikai Co., Ltd.) and depressurized by allowing to stand still for 1 minute at 19 Torr to 40 Torr. Subsequently, the valve was opened all at once to return to the normal atmospheric pressure. Processing such as cutting the roots was not carried out on the roots of the plants following vacuum infiltration treatment (Comparative Example 2, see FIG. 3A).

Cultivation after infection and measurement of GFP expression level were respectively carried out using the same methods described in 4 above and 5 above. The results are shown in Table 2.

### Example 3

The base of the roots of Nicotiana benthamiana obtained in Section 1 (4) above were damaged by repeatedly poking 10 times with a Dessert Fork 18-8 (Clip Inc.) on day 28 after seeding (see FIG. 3C). The plants were then each immediately (within 30 minutes after cutting) inverted and submerged in Agrobacterium bacterial solution (prepared in Section 2 (3) above) contained in a beaker so that the above ground portion was immersed in the solution. Subsequently, the beaker was placed in a vacuum desiccator (FV-3P, Tokyo Glass Kikai Co., Ltd.) and depressurized by allowing to stand still for 1 minute at 19 Torr to 40 Torr. Subsequently, the valve was opened all at once to return to the normal atmospheric pressure.

Cultivation after infection and measurement of GFP expression level were respectively carried out using the same methods described in Sections 4 and 5 above. The results are shown in Table 2.

TABLE 2

| | | GFP Expression Level |
|---|---|---|
| | Root Processing | per Leaf Fresh Weight |
| Comparative | Roots not processed | 100.0% |
| Example 2 | | |
| Example 3 | Base of roots pricked immediately | 116.3% |
| | before infection | |

The disclosure of Japanese Patent Application No. 2014-081110 (filing date: Apr. 10, 2014) is incorporated in the present specification by reference.

All references, patent applications and technical standards described in the present specification are incorporated herewith by reference to the same degree as the case of the incorporation of individual references, patent applications and technical standards by reference being specifically and individually described.

1. A method for producing a protein with a plant, comprising:
cultivating a plant capable of being infected with Agrobacterium using a hydroponic cultivation method,
infecting the plant grown using a hydroponic cultivation method by contacting with an infecting solution containing Agrobacterium having a polynucleotide encoding a target protein, and
expressing the target protein in the plant by further cultivating the plant after infection; wherein,
a damaging stimulus is imparted to the roots of the plant.

2. The method for producing a protein according to claim 1, wherein the damaging stimulus comprises cutting at least a portion of the roots of the plant.

3. The method for producing a protein according to claim 2, wherein the portion of the roots that is cut is 0.01% by mass to 50% by mass of the total fresh weight of the plant roots.

4. The method for producing a protein according to claim 2, wherein cutting of the roots is carried out prior to infecting.

5. The method for producing a protein according to claim 1, further comprising purifying and/or recovering the target protein from the plant after expressing.

6. The method for producing a protein according to claim 1, wherein the target protein is a medicinal protein.

7. The method for producing a protein according to claim 1, wherein the plant is Nicotiana benthamiana.

8. A method for producing a protein with a plant, comprising:
infecting the plant with Agrobacterium having a polynucleotide encoding a target protein, and
expressing the target protein in the plant by further cultivating the plant after infection; wherein,
a damaging stimulus is imparted to the roots of the plant.

9. The method for producing a protein according to claim 8, wherein the damaging stimulus is imparted to the roots of the plant prior to infection.

10. The method for producing a protein according to claim 8, wherein the damaging stimulus is imparted to the roots of the plant simultaneous to or after infection.
